(19) United States
(12) Patent Application Publication
(10) Pub. No.: US 2009/0286996 A1
(43) Pub. Date: Nov. 19, 2009

(54) PROCESS FOR THE PREPARATION OF ZILEUTON

(75) Inventors: Emanuele ATTOLINA, Palagiano (TA) (IT); Gianmaria Dell'Anna, Milano (IT); Roberto Rossi, Novara (IT); Pietro Allegrini, San Donato Milanese (IT); Gabriele Razzetti, Sesto San Giovanni (MI) (IT)

Correspondence Address: ROTHWELL, FIGG, ERNST & MANBECK, P.C., 1425 K STREET, N.W., SUITE 800, WASHINGTON, DC, 20005, US

(73) Assignee: Dipharma Francis S.r.I., Baranzate (MI) (IT)

(21) Appl. No.: 12/423,122

(22) Filed: Apr. 14, 2009

(30) Foreign Application Priority Data
May 15, 2008 (IT) MI 2008 A 000888

(51) Int. Cl. C07D 333/58; C07D 333/60; C07F 7/10

(52) U.S. Cl. 549/4; 549/58

(57) ABSTRACT

Process for preparing a compound of formula (I), or a salt thereof,

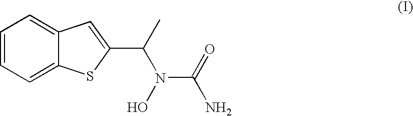

comprising reacting of a compound of formula (II)

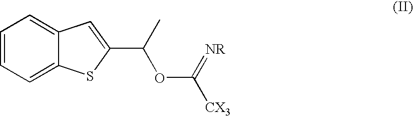

wherein X and R are as herein defined; with a compound of formula (III)

NH₂OZ (III)

wherein Z is a hydroxy protecting group, in presence of a catalyst, to obtain a compound of formula (IV), or a salt thereof,

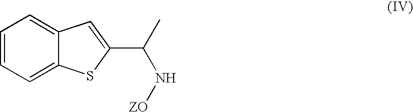

removing the hydroxyl protecting group to obtain a compound of formula (V), or a salt thereof;

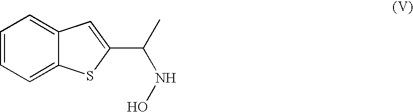

converting a compound of formula (V), or a salt thereof, into a compound of formula (I), or a salt thereof; and, if desired, converting a compound of formula (I) into a salt thereof, or vice versa.

### FIELD OF THE INVENTION

The present invention relates to a new process for the preparation of Zileuton

### TECHNOLOGICAL BACKGROUND

Zileuton, namely (±)-1-(1-benzo[b]thien-2-yl-ethyl)-1-hydroxyurea, having formula (I)

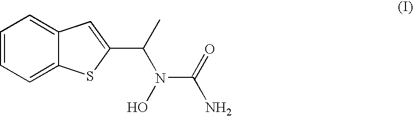

is a specific inhibitor of 5-lipoxygenase, used in the treatment of asthma.

EP 279 263 discloses its preparation by a process comprising the reduction of the double bond of an oxime having formula (A)

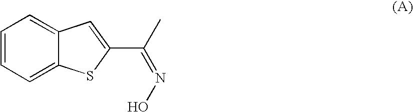

in the presence of a borane-pyridine complex, and the subsequent reaction of the product of formula (B) thus obtained

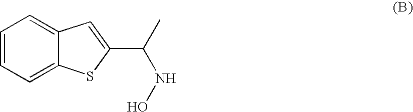

with trimethylsilylisocyanate (TMSNCO), or sodium or potassium cyanate in acid solution, or phosgene and ammonia. This process has several drawbacks. In particular borane is a toxic reagent and pyridine is a carcinogenic compound which limits the safety of the process.

Stewart and Brooks (A. O, Stewart and D. W. Brooks, J. Org. Chem. 1992, 57, 5020-5023) disclose an alternative process for the preparation of zileuton comprising the reaction of an alcohol having formula (C)

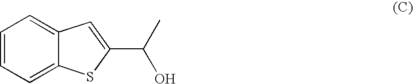

with N,O-bis(phenoxycarbonyl)-hydroxylamine in the presence of diisopropylazodicarboxylate (DIAD) and PPh₃ (Mitsunobu reaction), to obtain N,O-bis(phenoxycarbonyl)-N-(1-benzo[b]thien-2-yl-ethyl)-hydroxylamine, and the subsequent aminolysis. This process has several drawbacks from both safety and industrial application points of view, which limit its industrial application. Mitsunobu reaction brings to the formation of difficult to be eliminated by-products requiring, as a consequence, a cumbersome purification of the product by chromatography. Moreover, N,O-bis(phenoxycarbonyl)hydroxylamine is prepared using dichloroformate which is suspected to be mutagenic.

EP 589784 discloses another process for the preparation of Zileuton which comprises the reaction of a compound of formula (C), as defined above, with hydrobromic acid to obtain the corresponding halogenated compound 2-(bromoethyl)benzo[b]thiophene. Reacting this compound with O-benzyl-hydroxylamine, N-(benzyloxy)-N-(1-(benzo[b]thien-2-yl)ethyl)-urea is then obtained. This compound is further reacted with trimethylsilylisocyanate, and catalytic hydrogenation of the reaction product so obtained affords zileuton. This process has some drawbacks which limit its industrial application; in particular, the halogenation reaction is scarcely selective and with a poor yield, approximately of about 11%, with a really negative impact on the process costs.

U.S. Pat. No. 6,080,874 discloses a process for the preparation of zileuton in a single step, comprising reacting a compound of formula (C), as defined above, with hydroxyurea in presence of an acid.

At any rate this process also has some drawbacks since hydroxyurea is carcinogenic and in order to obtain a purer final product many hard-working purifications are necessary, thus limiting the safety and the industrial application of the process on industrial scale.

There is therefore the need of a new alternative process for the preparation of zileuton which makes use of easily available, non carcinogenic reagents or reagents suspected to be mutagenic or toxic, and which does not require hard-working purifications to obtain the final product.

### SUMMARY OF THE INVENTION

It has now been found that zileuton, namely (±)-1-(1-benzo[b]thien-2-yl-ethyl)-1-hydroxyurea, can be prepared according to a new process which allows to overcome the drawbacks of the state of the art.

Particularly the process of the invention allows preparing zileuton from easily available reagents which do not have particular safety problems from an industrial point of view, since they are not mutagenic or suspected to be carcinogenic.

Furthermore, the process according to the invention does not require cumbersome purifications in order to eliminate the by-products from the final product.

### DETAILED DESCRIPTION OF THE INVENTION

Object of the present invention is a process for the preparation of a compound of formula (I) or a salt thereof,

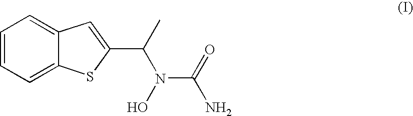

comprising reacting a compound of formula (II)

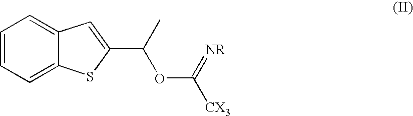

wherein each of X, being the same or different, is halogen, or two of them, being the same or different, are halogen and the remaining one is a cyano group; and R is hydrogen or a C₁-C₆ alkyl, aryl or aryl-C₁-C₆ alkyl group; with a compound of formula (III)

NH₂OZ (III)

wherein Z is a hydroxy protecting group, in presence of a catalyst, to obtain a compound of formula (IV), or a salt thereof,

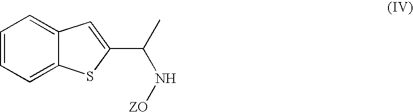

wherein Z is as defined above;

removing the hydroxyl protecting group in a compound of formula (IV) to obtain a compound of formula (V), or a salt thereof;

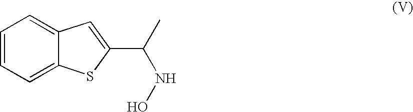

converting a compound of formula (V), or a salt thereof, into a compound of formula (I), or a salt thereof, and, if desired, converting a compound of formula (I) into a salt thereof, or vice versa.

A C₁-C₆ alkyl group is preferably a C₁-C₄ straight or branched alkyl group, in particular methyl, ethyl, propyl or t-butyl.

An aryl group is preferably phenyl or naphtyl, in particular phenyl.

An aryl-C₁-C₆ alkyl group is preferably an aryl-C₁-C₄ alkyl group, in particular benzyl or phenylethyl.

A halogen is for example chlorine, fluorine, bromine, in particular chlorine.

A catalyst can be a sulfonic acid, such as benzensulfonic acid, p-toluensolfonic acid or camphorsulfonic acid or one of the “acid washed molecular sieves”, for example AW 300 MS®. More typically, a catalyst is a Lewis acid catalyst, for example an organic Lewis acid such as a tri(C₁-C₆) alkylsilyl or phenyl-C₁-C₆ alkylsilyl trifluoromethansulfonate, for example trimethylsilyl-trifluoromethansulfonate, t-butyldimethylsilyl-trifluoromethansulfonate, triisopropylsilyl-trifluoromethansulfonate, t-butyldiphenylsilyl-trifluoromethansulfonate, or an inorganic Lewis acid, such as boron trifluoride etherate, zinc iodide, bismuth or scandium trifluoromethansulfonate or a lanthanide trifluoromethansulfonate, in particular lanthanum or ytterbium. Preferably the catalyst is trimethylsilyl-trifluoromethansulfonate.

The amount of the catalyst compared to the compound of formula (II) can be approximately comprised between 0.01 and 0.3 moles/mole, preferably about 0.05-0.15 moles/mole.

A hydroxy protecting group can be for example one of the protecting groups used in the chemistry of alcohols, for example a tri(C₁-C₆) alkyl-silyl, for example trimethylsilyl, t-butyl-dimethylsilyl, benzyl, phenylethyl, naphthalenylmethyl, preferably trimethylsilyl.

When in a compound of formula (III) the protecting group of the hydroxyl moiety is a trimethylsilyl group, the reaction between a compound of formula (II), or a salt thereof, and a compound of formula (III) can directly provide a compound of formula (V), without isolating the protected intermediate of formula (IV).

A compound of formula (IV), which can be isolated, is converted in a compound of formula (V) by deprotecting the hydroxyl moiety according to known methods, for example by hydrolysis or by catalytic hydrogenation.

The reaction between a compound of formula (II), or a salt thereof, and a compound of formula (III), is preferably carried out in a solvent selected for example from a dipolar aprotic solvent, typically dimethylformamide, dimethylacetamide, acetonitrile, dimethylsulfoxide; an ether, for example diethylether, methyl-t-butyl-ether, tetrahydrofuran, or dioxane; a chlorinated solvent, for example dichloromethane, chloroform or chlorobenzene; an apolar solvent, such as an aliphatic hydrocarbon, for example hexane or cyclohexane, or an aromatic hydrocarbon such as benzene or toluene; an ester, for example ethyl or methyl acetate; or a ketone, for example acetone, methylethylketone, methylisobutylketone, or a mixture of two or more, preferably two or three, of said solvents. Such solvent is preferably a chlorinated solvent, in particular dichloromethane or an aromatic hydrocarbon in particular toluene.

A compound of formula (II), as defined above, is a new compound and is a further object of the invention.

Preferred examples of compounds of formula (II) are:
1-(1-benzo[b]thien-2-yl-ethyl)-2-trichloroacetimidate,
1-(1-benzo[b]thien-2-yl-ethyl)-2-cyano-2-dichloroacetimidate, and
1-(1-benzo[b]thien-2-yl-ethyl)-N-phenyl-2-trifluoroacetimidate.

The compounds of formula (IV), and the salts thereof, wherein Z, being as defined above, is different from benzyl, are new compounds and further object of this invention.

Preferred examples of compounds of formula (IV) are:
O-trimethylsilyl-1-(1-benzo[b]thien-2-yl-ethyl)-1-hydroxylamine, O-t-butyl-dimethylsilyl-1-(1-benzo[b]thien-2-yl-ethyl)-1-hydroxylamine, O-phenylethyl-1-(1-benzo[b]thien-2-yl-ethyl)-1-hydroxylamine, and O-naphtalenylmethyl-1-(1-benzo[b]thien-2-yl-ethyl)-1-hydroxylamine.

The conversion of a compound of formula (V), or a salt thereof, into a compound of formula (I), or a salt thereof, can be carried out for example by reacting a compound of formula (V) with trimethylsilyl isocyanate or by treatment with sodium or potassium cyanate in acid solution, as disclosed by EP 279 263.

Analogously, the conversion of a compound of formula (I) in a salt thereof, or vice versa, can be carried out according to known methods.

A compound of formula (II), as defined above, can be prepared by a process comprising the reaction of a compound of formula (VI), or a salt thereof,

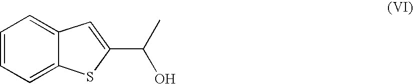

with a compound of formula (VII) or (VIII), in presence of a base,

CX₃C(═NR₁)R₂ (VII)

CX₃C≡N (VIII)

wherein R₁ is a C₁-C₆ alkyl, aryl or aryl-C₁-C₆ alkyl group, preferably phenyl; R₂ is halogen, preferably chlorine; and X is as defined above.

When the reaction is carried out with a compound of formula CX₃C≡N, a compound of formula (II) wherein R is hydrogen is obtained.

A base can be an organic base, such as a tertiary amine, for example 1,8-diazabicyclo[5.4.0]undec-7-ene (DBU), 1,5-diazabicyclo[4.3.0]non-5-ene (DBN), 1,4-diazabicyclo[2.2.2]octane (DABCO), diisopropylethylamine; or triphenylphosphine; or an inorganic base such as, for example, sodium hydride; a C₁-C₆ alkoxide, for example sodium methoxide, ethoxide or t-butoxide; or a carbonate of an alkaline metal, in particular potassium or cesium.

The reaction between a compound of formula (VI) and a compound of formula (VII) or (VIII) is preferably carried out in presence of a solvent, typically an organic solvent, selected for example among those cited above; or a mixture of two or more, preferably two or three, of said solvents. The reaction is preferably carried out in a chlorinated solvent, in particular dichloromethane or in an aromatic hydrocarbon for example toluene.

In the reaction between a compound of formula (VI) and a compound of formula (VII) the amount of base used can be approximately stoichiometric, preferably the base is potassium carbonate; whereas in the reaction between a compound of formula (VI) and a compound of formula (VIII), the amount of base used is preferably in a catalytic amount, and the base is preferably sodium hydride or diazabicycloundecene.

Preferred examples of compounds of formula (VII) and (VIII) are:

CCl_{3l C≡N, CCl}₂(C≡N)₂ and CF₃C(═NPh)Cl

wherein Ph is phenyl.

The compound of formula (I), namely zileuton, or a salt thereof, obtained according to the new method of the invention has a purity equal to or higher than 99.5% (HPLC), therefore complying with the Regulatory requirements for pharmaceutical products. If desired, it can be subsequently purified according to known methods, for example by one or more recrystallizations to obtain a purity equal to or higher than 99.9% (HPLC).

A compound of formula (I), namely zileuton, with such a high purity degree has never been disclosed before, therefore it is new and is a further object of this invention.

The particle size of the crystals of zileuton, obtained according to the invention, is characterized by a D₅₀ value comprised between about 25 and 250 μm, wherein D₅₀ is a particle diameter so as to 50% (in volume) of the particle sample has a diameter equal to or lower than the specific value. Such value, if desired, can be reduced by micronization or fine grinding, or can be increased controlling the crystallization conditions, for example by slow cooling of the solution, as it is known in the art.

According to the present invention, with the term compound of formula (I), (IV), (V) and (VI) the same compound or a salt thereof is meant, in particular a pharmaceutical acceptable salt thereof with an acid or a base. Typically one among those commonly used in the art, for example succinate, sulfate, chloridrate, acetate, formate, propionate, mesylate, or sodium, potassium ammonium salts.

The following examples illustrate the invention.

### Example 1

### Preparation of 1-(1-benzo[b]thien-2-yl-ethyl)-2-trichloroacetimidate, (II, R═H, X═Cl)

In a 1 L four-necked flask provided with a mechanic stirrer, thermometer, reflux condenser, dropping funnel and under nitrogen inert atmosphere, 150 ml of dichloromethane are loaded, 2-(hydroxyethyl)benzo[b]thiophene (50 g), of formula (VI), and CCl₃CN (48.5 g). The solution is maintained under stirring at room temperature (20-25° C.) till its complete dissolution, thereafter cooled to a temperature of 0-5° C. A solution obtained dissolving 1,8-diazabicyclo[5.4.0]undec-7-ene (0.85 g) in dichloromethane (2 ml) is slowly dropwise added maintaining the temperature below about 10° C. At the end of the addition the temperature of the reaction mixture is brought to about 20-25° C. and maintained under stirring for about 1 hour. The reaction mixture is directly used in the following step. Part of the solution has been isolated and analyzed:

NMR assay higher than 98%.

¹H NMR (300 MHz, CDCl₃), ppm: 8.42 (s, 1H), 7.75 (d, 1H, J 10.8 Hz) 7.70 (d, 1H, J 8.7 Hz), 7.32-7.24 (m, 3H), 6.32 (q, 1H, J 6.3 Hz), 1.78 (d, 3H, J 6.3 Hz).

In a similar way 1-(1-benzo[b]thien-2-yl-ethyl)-2-cyan-2-dichloro-acetimidate can be obtained.

### Example 2

### Preparation of 1-(1-benzo[b]thien-2-yl-ethyl)-N-phenyl-2-trifluoroacetimidate (II, R═Ph, X═F)

In a 1 L four-necked flask provided with a mechanic stirrer, thermometer, reflux condenser, dropping funnel and under nitrogen inert atmosphere 200 ml of tetrahydrofuran, 2-(hydroxyethyl)benzo[b]tiophene (50 g) and (N-phenyl)trifluoroacetimidoyl chloride (64.0 g) are added. The solution is maintained under stirring at about 0-5° C., then potassium carbonate (46.5 g) is added.

The reaction is stirred for 1 hour and then filtered off. The solution can be used in the subsequent steps without isolating the obtained product.

### Example 3

### Preparation of O-trimethylsilyl-1-(1-benzo[b]thien-2-yl-ethyl)-1-hydroxylamine (IV)

The reaction mixture obtained in Example 1 is cooled to about 0-5° C., then a dichloromethane solution containing 14.5% (244 g) of trimethylsilyl hydroxylamine is slowly dropwise added, checking that the temperature remains in the range of 5-10° C. After the addition a solution of trimethylsilyl-trifluoromethansulfonate (3.1 g) in dichloromethane is added. The mixture is stirred for 1 hour, and the inner temperature is maintained at about 20° C., then 2.0 g of triethylamine are added.

The mixture is concentrated under reduced pressure and the residue is treated with toluene (200 ml). The suspension is filtered off to remove trichloroacetamide and the organic solution is concentrated at reduced pressure providing the crude product with a NMR assay higher than 80%.

¹H NMR (300 MHz, CDCl₃), ppm: 7.82-7.79 (m, 1H) 7.74-7.71 (m, 1H), 7.36-7.26 (m, 2H), 7.21 (s, 1H), 5.18 (bs, 1H), 4.40 (q, 1H, J 6.4 Hz), 1.56 (d, 3H, J 6.4 Hz), 0.17 (s, 9H).

In a similar way and using the suitable protected hydroxylamines:

O-t-butyl-dimethylsilyl-1-(1-benzo[b]thien-2-yl-ethyl)-1-ydroxylamine, O-phenylethyl-1-(1-benzo[b]thien-2-yl-ethyl)-1-hydroxylamine, and O-naphtalenylmethyl-1-(1-benzo[b]thien-2-yl-ethyl)-1-hydroxylamine can be obtained.

### Example 4

### Preparation of 1-(1-benzo[b]thien-2-yl-ethyl)-1-hydroxylamine (V)

The toluene solution of example 3 obtained after filtration of trichloroacetamide is treated in a flask with water (200 ml) and acidified with 36% HCl till pH 2. The phases are separated and the aqueous one is diluted with isopropanol (20 ml) and neutralized by slow dropwise addition of 20% NaOH. The suspension is stirred for 1 hour at 25° C. and then cooled to 0° C. and filtered off. The solid is washed with H₂O (50 ml) and dried. About 42 g of 1-(1-benzo[b]thien-2-yl-ethyl)-1-hydroxylamine with HPLC assay higher than 98% are obtained and with a 78% yield starting from 2-(hydroxyethyl)benzo[b]thiophene. M.P. 83-84° C.

¹H NMR (300 MHz, CDCl₃), ppm: 7.81-7.78 (m, 1H) 7.72-7.69 (m, 1H), 7.35-7.24 (m, 2H), 7.20 (s, 1H), 5.54 (bs, 2H), 4.48 (q, 1H, J 6.6 Hz), 1.53 (d, 3H, J 6.6 Hz).

### Example 5

### Preparation of Zileuton Starting from 1-(1-benzo[b]thien-2-yl-ethyl)-1-hydroxylamine (V)

In a 2 L four-necked flask provided with a mechanic stirrer, thermometer, reflux condenser, dropping funnel 1-(1-benzo[b]thien-2-yl-ethyl)-1-hydroxylamine (50 g), potassium cyanate (42 g), water (50 ml), ethyl acetate (600 ml) are loaded. The suspension is stirred for 1 hour at 20° C. and then 3.6% HCl (350 ml) is slowly dropwise added. The mixture is stirred at 25° C. for two hours and then the solid is filtered off and washed with ethyl acetate and water. After drying 55 g of zileuton having a HPLC assay higher than 99.5% are obtained.

### Example 6

### Preparation of Zileuton Starting from 2-(hydroxyethyl)benzo[b]thiophene (VI)

In a reactor under nitrogen inert atmosphere dichloromethane (83.7 kg), 2-(hydroxyethyl)benzo[b]thiophene (20.5 kg) and CCl₃CN (19.9 kg) are loaded. The solution is cooled to a temperature of 0-5° C. A solution obtained dissolving 1,8-diazabicyclo[5.4.0]undec-7-ene (350 g) in dichloromethane (540 g) is added, maintaining the temperature below about 10° C. At the end of the addition, the reaction mixture is brought to a temperature of about 0-5° C. and stirred for about one hour. Then a 14.5% (100 g) solution of trimethylsilyl hydroxylamine in dichloromethane is slowly dropwise added, checking that the temperature remains at about 0-5° C.

After the addition, a solution obtained dissolving trimethylsilyl-trifluoromethansulfonate (1.2 kg) in dichloromethane (1.6 kg) is dropwise added. The mixture is stirred for two hours, checking that the inner temperature remains at about 10° C. Subsequently 1 kg of triethylamine is added. The mixture is concentrated under reduced pressure and the residue is treated with toluene (130 kg). The suspension is filtered and the toluene solution is treated with water (831) and basified with 30% NaOH. The heterogeneous mixture is maintained under stirring for 24 hours at a temperature of about 25° C. The phases are separated and the organic phase is washed with water. A solution obtained by dissolving potassium cyanate (18.2 kg) in water (901) is then added and subsequently 36% HCl (34 kg) is dropwise added. The mixture is stirred at about 25° C. for 18 hours then the solid is filtered off and washed with toluene and water. After drying 17.5 kg of zileuton are obtained, having a HPLC assay higher than 99.5%.

1. Process for preparing a compound of formula (I), or a salt thereof, 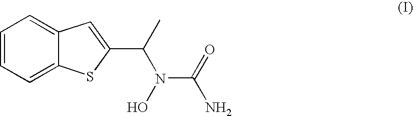
comprising reacting a compound of formula (II) 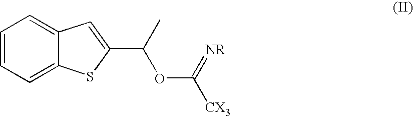
wherein each of X, being the same or different, is halogen, or two of them, being the same or different, are halogen and the remaining one is a cyano group; and R is hydrogen or a C₁-C₆ alkyl, aryl or aryl-C₁-C₆ alkyl group; with a compound of formula (III)
NH₂OZ (III)
wherein Z is a hydroxy protecting group, in presence of a catalyst, to obtain a compound of formula (IV), or a salt thereof, 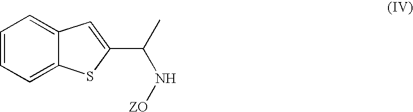
wherein Z is as defined above;
removing the hydroxyl protecting group in a compound of formula (IV) to obtain a compound of formula (V), or a salt thereof, 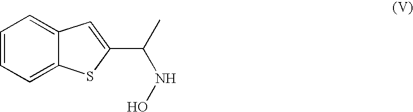
converting a compound of formula (V), or a salt thereof, into a compound of formula (I), or a salt thereof, and, if desired, converting a compound of formula (I) into a salt thereof, or vice versa.

2. Process according to claim 1, wherein the catalyst is a sulfonic acid or a Lewis acid.

3. Process according to claim 2, wherein the catalyst is selected among benzensulfonic acid, p-toluensulfonic acid, camphorsulfonic acid, one of the “acid washed molecular sieves”, a tri(C₁-C₆) alkylsilyl or phenyl-C₁-C₆ alkylsilyl trifluoromethansulfonate, borontrifluoride-etherate, zinc iodide, bismuth or scandium trifluoromethansulfonate or a lanthanide trifluoromethansulfonate.

4. Process according to claim 3 wherein the catalyst is selected from trimethyl silyl-trifluoromethansulfonate, t-butyldimethylsilyl-trifluoromethansulfonate, triisopropylsilyl-trifluoromethansulfonate and t-butyldiphenylsilyl-trifluoromethansulfonate.

5. Process according to claim 1 wherein the amount of catalyst to the compound of formula (II) is approximately comprised between 0.01 and 0.3 moles/mole.

6. Process according to claim 1, wherein the protecting group Z is a tri (C₁-C₆) alkyl-silyl group.

7. Process according to claim 1, wherein the protected intermediate of formula (IV) is not isolated.

8. Process according to claim 1, wherein a compound of formula (II), or a salt thereof, is prepared by a process comprising the reaction of a compound of formula (VI), or a salt thereof 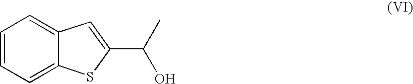
with a compound of formula (VII) or (VIII), in the presence of a base,
CX₃C(═NR₁)R₂ (VII)
CX₃C≡N (VIII)
wherein R₁ is a C₁-C₆ alkyl, aryl o aryl-C₁-C₆ alkyl group; R₂ is halogen; and X is as defined in claim 1.

9. Process according to claim 8 wherein the base is selected among a tertiary amine, triphenylphosphine, sodium hydride, a C₁-C₆ alkoxide or an alkaline metal carbonate.

10. Process according to claim 9 wherein the base is selected between sodium hydride and diazabicycloundecene.

11. A compound of formula (II) 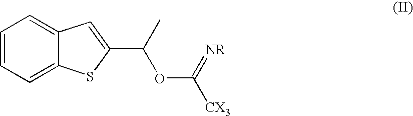
wherein each of X, being the same or different, is halogen, or two of them, being the same or different, are halogen and the remaining one is cyano; and R is hydrogen or a C₁-C₆ alkyl, aryl or aryl-C₁-C₆ alkyl group.

12. A compound of formula (II), according to claim 11, which is:
1-(1-benzo[b]thien-2-yl-ethyl)-2-trichloroacetimidate,
1-(1-benzo[b]thien-2-yl-ethyl)-2-cyan-2-dichloroacetimidate, or
1-(1-benzo[b]thien-2-yl-ethyl)-N-phenyl-2-trifluoroacetimidate.

13. A compound of formula (IV), or a salt thereof 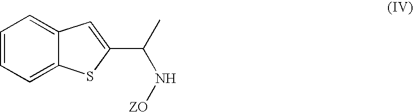
wherein Z is a hydroxy protecting group, except benzyl group.

14. A compound of formula (IV), according to claim 13, which is:
O-trimethylsilyl-1-(1-benzo[b]thien-2-yl-ethyl)-1-hydroxylamine,
O-t-butyl-dimethysilyl-1-(1-benzo[b]thien-2-yl-ethyl)-1-hydroxylamine,
O-phenylethyl-1-(1-benzo[b]thien-2-yl-ethyl)-1-hydroxylamine, or
O-naphtalenylmethyl-1-(1-benzo[b]thien-2-yl-ethyl)-1-hydroxylamine.

15. Zileuton having a purity equal to or higher than 99.5%.
